(19) United States
(12) Patent Application Publication
(10) Pub. No.: US 2021/0015385 A1
(43) Pub. Date: Jan. 21, 2021

(54) SYSTEMS AND METHODS FOR FREQUENCY AND WIDE-BAND TAGGING OF MAGNETOENCEPHALOGRAPY (MEG) SIGNALS

(71) Applicant: HI LLC, Los Angeles, CA (US)

(72) Inventors: Husam Katnani, Braintree, MA (US); Antonio Lara, Sherman Oaks, CA (US); Arnulf Graf, Pasadena, CA (US); Julien Dubois, Santa Monica, CA (US)

(21) Appl. No.: 16/928,810

(22) Filed: Jul. 14, 2020

Related U.S. Application Data
(60) Provisional application No. 62/898,461, filed on Sep. 10, 2019.
(60) Provisional application No. 62/874,887, filed on Jul. 16, 2019.

(51) Int. Cl. A61B 5/04; G06F 3/01

(52) U.S. Cl. CPC A61B 5/04009; G06F 3/015

(57) ABSTRACT

A biological signal detection system includes one or more magnetic field sensors for placement on a user and for detecting signals generated by biological magnetic field sources of the user; at least one memory; at least one processor coupled to the at least one memory and the one or more magnetic field sensors and configured to receive the detected signals of the one or more magnetic field sensors. The at least one processor is configured to perform actions including receiving the detected signals from the magnetic field sensors; determining whether the detected signals are modulated by a predetermined stimulus tag; and, in response to the determination, identifying which of the detected signals are modulated by the predetermined stimulus tag.

### CROSS-REFERENCE TO RELATED APPLICATIONS

This application claims the benefit of U.S. Provisional Patent Application Ser. Nos. 62/874,887, filed Jul. 16, 2019, and 62/898,461, filed Sep. 10, 2019, both of which are incorporated herein by reference in their entireties.

### FIELD

The present disclosure is directed to the area of magnetic field measurement systems including systems for magnetoencephalography (MEG). The present disclosure is also directed to methods and systems for tagging MEG signals and employing the tagged MEG signals in applications.

### BACKGROUND

In the nervous system, neurons propagate signals via action potentials. These are brief electric currents which flow down the length of a neuron causing chemical transmitters to be released at a synapse. The time-varying electrical currents within an ensemble of neurons generate a magnetic field. Magnetoencephalography (MEG), the measurement of magnetic fields generated by the brain, is one method for observing these neural signals.

### BRIEF SUMMARY

One embodiment is a biological signal detection system, including one or more magnetic field sensors for placement on a user and for detecting signals generated by biological magnetic field sources of the user; at least one memory; at least one processor coupled to the at least one memory and the one or more magnetic field sensors and configured to receive the detected signals of the one or more magnetic field sensors. The at least one processor is configured to perform actions including receiving the detected signals from the magnetic field sensors; determining whether the detected signals are modulated by a predetermined stimulus tag; and, in response to the determination, identifying which of the detected signals are modulated by the predetermined stimulus tag.

Another embodiment is a non-transitory computer-readable medium having stored thereon instructions for execution by a processor to perform actions including: receiving detected signals from one or more magnetic field sensors; determining whether the detected signals are modulated by a predetermined stimulus tag; and, in response to the determination, identifying which of the detected signals are modulated by the predetermined stimulus tag.

In at least some embodiments, the actions further include producing a stimulus for the user; and modulating a portion of the stimulus using the predetermined stimulus tag. In at least some embodiments, the actions further include altering the stimulus in response to identifying which of the detected signals are modulated by the predetermined stimulus tag. In at least some embodiments, altering the stimulus includes altering a visual content of the stimulus in response to the determination.

In at least some embodiments the biological signal detection system is a closed-loop MEG-brain machine interface.

In at least some embodiments, the stimulus is a visual stimulus. In at least some embodiments, the stimulus is an audible stimulus. In at least some embodiments, the stimulus is a tactile stimulus.

In at least some embodiments, the stimulus tag is a frequency tag at a predetermined frequency. In at least some embodiments, the stimulus tag is a broadband tag over a predetermined range of frequencies. In at least some embodiments, the stimulus tag is a noise tag.

In at least some embodiments, determining whether the detected signals are modulated by the predetermined stimulus tag includes generating a power spectrum of the detected signals and determining whether the detected signals are modulated by the predetermined stimulus tag using the power spectrum. In at least some embodiments, determining whether the detected signals are modulated by the predetermined stimulus tag comprises generating a correlation between the detected signals and the predetermined stimulus tag and determining whether the detected signals are modulated by the predetermined stimulus tag. In at least some embodiments, determining whether the detected signals are modulated by the predetermined stimulus tag includes time series correlation of the detected signal to the determined stimulus tag.

In at least some embodiments, determining whether the detected signals are modulated by the predetermined stimulus tag includes determining whether the detected signals are modulated by a predetermined first stimulus tag; wherein identifying which of the detected signals are modulated by the predetermined stimulus tag includes identifying which of the detected signals are modulated by the predetermined first stimulus tag; wherein the actions further include determining whether the detected signals are modulated by a predetermined second stimulus tag, wherein the second stimulus tag is different from the first stimulus tag; and, in response to the determination, identifying which of the detected signals are modulated by the predetermined second stimulus tag.

In at least some embodiments, the actions further include communicating with a stimulus generator indicating which of the detected signals are modulated by the predetermined stimulus tag. In at least some embodiments, the detected signals originate in a brain of a user.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic block diagram of one embodiment of a magnetic field measurement system, according to the invention;

FIG. 1B is a schematic block diagram of one embodiment of a magnetometer, according to the invention;

FIG. 1C is a schematic block diagram of one embodiment of an environment for biological signal detection, according to the invention;

FIG. 2 shows a magnetic spectrum with lines indicating dynamic ranges of magnetometers operating in different modes;

FIG. 3 is a graph of the power spectral density (PSD) of spatially-filtered MEG data obtained in response to visual stimulation modulated by a frequency tag at three different frequencies, according to the invention;

FIG. 4 is a confusion matrix for single-trial decoding of 10 second visual tags, according to the invention;

FIG. 5 is a graph of decoding accuracy for the spatially-filtered MEG data obtained in response to visual stimulation modulated by a frequency tag at three different frequencies, according to the invention;

FIG. 6 is a confusion matrix for single-trial decoding of 2 second visual frequency and noise tags, according to the invention; and

FIG. 7 is a flowchart of one embodiment of a method of identifying biological signals that are modulated by one or more predetermined stimulus tags, according to the invention.

### DETAILED DESCRIPTION

The present disclosure is directed to the area of magnetic field measurement systems including systems for magnetoencephalography (MEG). The present disclosure is also directed to methods and systems for tagging MEG signals and employing the tagged MEG signals in applications.

Herein the terms “ambient background magnetic field” and “background magnetic field” are interchangeable and used to identify the magnetic field or fields associated with sources other than the magnetic field measurement system and the magnetic field sources of interest, such as biological source(s) (for example, neural signals from a user's brain) or non-biological source(s) of interest. The terms can include, for example, the Earth's magnetic field, as well as magnetic fields from magnets, electromagnets, electrical devices, and other signal or field generators in the environment, except for the magnetic field generator(s) that are part of the magnetic field measurement system.

The terms “gas cell”, “vapor cell”, and “vapor gas cell” are used interchangeably herein. Below, a vapor cell containing alkali metal vapor is described, but it will be recognized that other vapor cells can contain different gases or vapors for operation.

The methods and systems are described herein using optically pumped magnetometers (OPMs), but it will be understood that other magnetic field measurement devices, such as SQUIDs, can be used as an alternative to, or in addition to, OPMs. While there are many types of OPMs, in general magnetometers operate in two modalities: vector mode and scalar mode. In vector mode, the OPM can measure one, two, or all three vector components of the magnetic field; while in scalar mode the OPM can measure the total magnitude of the magnetic field.

Vector mode magnetometers measure a specific component of the magnetic field, such as the radial and tangential components of magnetic fields with respect the scalp of the human head. Vector mode OPMs often operate at zero-field and may utilize a spin exchange relaxation free (SERF) mode to reach femto-Tesla sensitivities. A SERF mode OPM is one example of a vector mode OPM, but other vector mode OPMs can be used at higher magnetic fields. These SERF mode magnetometers can have high sensitivity but may not function in the presence of magnetic fields higher than the linewidth of the magnetic resonance of the atoms of about 10 nT, which is much smaller than the magnetic field strength generated by the Earth.

Magnetometers operating in the scalar mode can measure the total magnitude of the magnetic field. (Magnetometers in the vector mode can also be used for magnitude measurements.) Scalar mode OPMs often have lower sensitivity than SERF mode OPMs and are capable of operating in higher magnetic field environments.

The magnetic field measurement systems, such as a biological signal detection system, described herein can be used to measure or observe electromagnetic signals generated by one or more magnetic field sources (for example, neural signals or other biological sources) of interest. The system can measure biologically generated magnetic fields and, at least in some embodiments, can measure biologically generated magnetic fields in an unshielded or partially shielded environment. Aspects of a magnetic field measurement system will be exemplified below using magnetic signals from the brain of a user; however, biological signals from other areas of the body, as well as non-biological signals, can be measured using the system. In at least some embodiments, the system can be a wearable MEG system that can be used outside a magnetically shielded room.

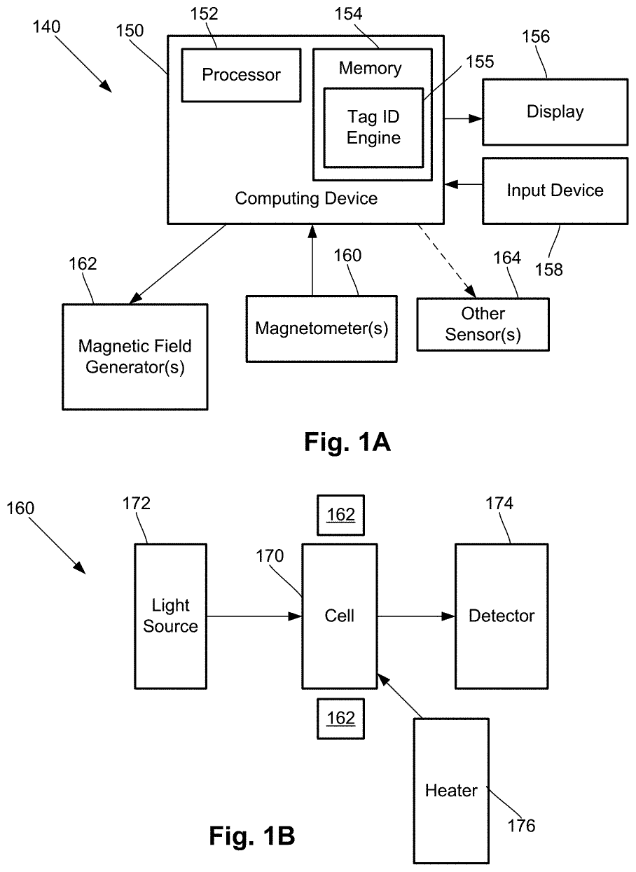

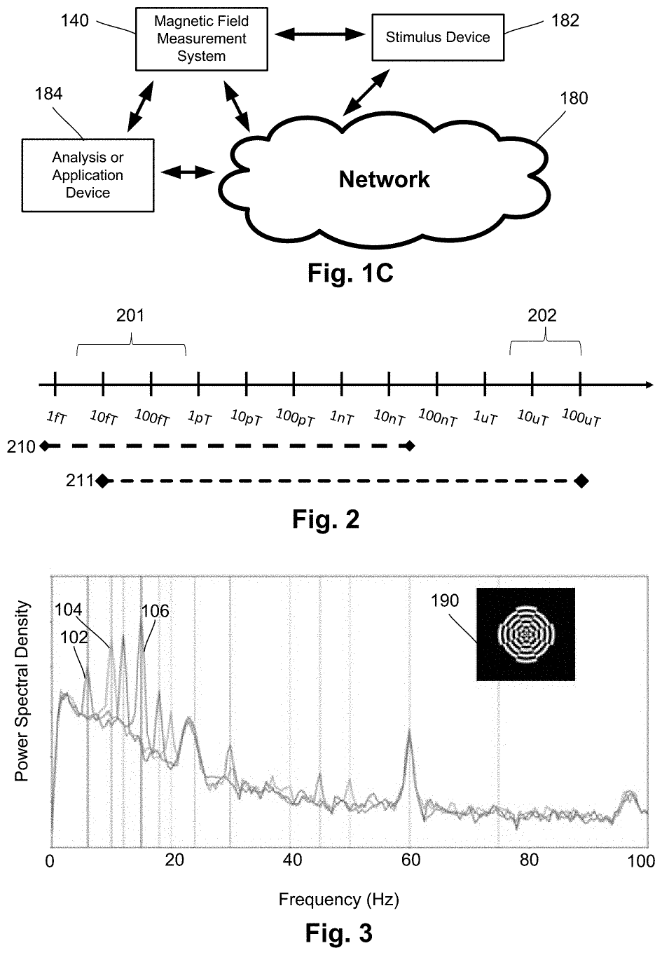

A magnetic field measurement system, such as a biological signal detection system, can utilize one or more magnetic field sensors. Magnetometers will be used herein as an example of magnetic field sensors, but other magnetic field sensors may also be used. FIG. 1A is a block diagram of components of one embodiment of a magnetic field measurement system 140 (such as a biological signal detection system.) The system 140 can include a computing device 150 or any other similar device that includes a processor 152, a memory 154, a display 156, an input device 158, one or more magnetometers 160 (for example, an array of magnetometers) which can be OPMs, one or more magnetic field generators 162, and, optionally, one or more other sensors 164 (e.g., non-magnetic field sensors). The system 140 and its use and operation will be described herein with respect to the measurement of neural signals arising from one or more magnetic field sources of interest in the brain of a user as an example. It will be understood, however, that the system can be adapted and used to measure signals from other magnetic field sources of interest including, but not limited to, other neural signals, other biological signals, as well as non-biological signals.

The computing device 150 can be a computer, tablet, mobile device, field programmable gate array (FPGA), microcontroller, or any other suitable device for processing information or instructions. The computing device 150 can be local to the user or can include components that are non-local to the user including one or both of the processor 152 or memory 154 (or portions thereof). For example, in at least some embodiments, the user may operate a terminal that is connected to a non-local computing device. In other embodiments, the memory 154 can be non-local to the user.

The computing device 150 can utilize any suitable processor 152 including one or more hardware processors that may be local to the user or non-local to the user or other components of the computing device. The processor 152 is configured to execute instructions such as instructions provided as part of a tag identification (ID) engine 155 stored in the memory 154.

Any suitable memory 154 can be used for the computing device 150. The memory 154 illustrates a type of computer-readable media, namely computer-readable storage media. Computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 156 can be any suitable display device, such as a monitor, screen, or the like, and can include a printer. In some embodiments, the display is optional. In some embodiments, the display 156 may be integrated into a single unit with the computing device 150, such as a tablet, smart phone, or smart watch. In at least some embodiments, the display is not local to the user. The input device 158 can be, for example, a keyboard, mouse, touch screen, track ball, joystick, voice recognition system, or any combination thereof, or the like. In at least some embodiments, the input device is not local to the user.

The magnetic field generator(s) 162 can be, for example, Helmholtz coils, solenoid coils, planar coils, saddle coils, electromagnets, permanent magnets, or any other suitable arrangement for generating a magnetic field. As an example, the magnetic field generator 162 can include three orthogonal sets of coils to generate magnetic fields along three orthogonal axes. Other coil arrangements can also be used. The optional sensor(s) 164 can include, but are not limited to, one or more position sensors, orientation sensors, accelerometers, image recorders, or the like or any combination thereof.

The one or more magnetometers 160 can be any suitable magnetometer including, but not limited to, any suitable optically pumped magnetometer. Arrays of magnetometers are described in more detail herein. In at least some embodiments, at least one of the one or more magnetometers (or all of the magnetometers) of the system is arranged for operation in the SERF mode. Examples of magnetic field measurement systems or methods of making such systems or components for such systems are described in U.S. Patent Application Publications Nos. 2020/0072916; 2020/0056263; 2020/0025844; 2020/0057116; 2019/0391213; 2020/0088811; 2020/005711; 2020/0109481; and 2020/0123416; U.S. patent application Ser. Nos. 16/573,394; 16/573,524; 16/679,048; 16/741,593; 16/752,393; 16/850,380 and 16/850,444, and U.S. Provisional Patent Application Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; and 62/983,406, all of which are incorporated herein by reference in their entireties. In at least some embodiments, instead of, or in addition to, OPMs, other magnetometers or magnetic field sensors, such as SQUIDs, can be used.

FIG. 1B is a schematic block diagram of one embodiment of a magnetometer 160 which includes a vapor cell 170 (also referred to as a “cell” or a “gas cell”) such as an alkali metal vapor cell; a heating device 176 to heat the cell 170; a light source 172; and a detector 174. In addition, coils of a magnetic field generator 162 can be positioned around the vapor cell 170. The vapor cell 170 can include, for example, an alkali metal vapor (for example, rubidium in natural abundance, isotopically enriched rubidium, potassium, or cesium, or any other suitable alkali metal such as lithium, sodium, or francium) and, optionally, one, or both, of a quenching gas (for example, nitrogen) or a buffer gas (for example, nitrogen, helium, neon, or argon). In some embodiments, the vapor cell may include the alkali metal atoms in a prevaporized form prior to heating to generate the vapor.

The light source 172 can include, for example, a laser to, respectively, optically pump the alkali metal atoms and probe the vapor cell. The light source 172 may also include optics (such as lenses, waveplates, collimators, polarizers, and objects with reflective surfaces) for beam shaping and polarization control and for directing the light from the light source to the cell and detector. Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source. In some embodiments, the light source 172 may include two light sources: a pump light source and a probe light source.

The detector 174 can include, for example, an optical detector to measure the optical properties of the transmitted probe light field amplitude, phase, or polarization, as quantified through optical absorption and dispersion curves, spectrum, or polarization or the like or any combination thereof. Examples of suitable detectors include, but are not limited to, a photodiode, charge coupled device (CCD) array, CMOS array, camera, photodiode array, single photon avalanche diode (SPAD) array, avalanche photodiode (APD) array, or any other suitable optical sensor array that can measure the change in transmitted light at the optical wavelengths of interest.

FIG. 1C illustrates one embodiment for an environment for determining and identifying tagged magnetic field signals, as described in more detail below. The environment includes the magnetic field measurement system 140 (for example, a biological signal detection system) of FIG. 1. The environment can also include a stimulus device 182 which provides a stimulus (for example, a visual, auditory, or tactile stimulus) where at least a portion of the stimulus can be tagged, as described below, using, for example, a tagging engine. In other embodiments, the computing device 150 of the magnetic field measurement system 140 can also be the stimulus device 182. In some embodiments, a separate analysis or application device 184 can also be included and can receive detected biological signals or analyzed biological signals or the like from the magnetic field measurement system 140 and may also communicate (or include) the stimulus device 182.

The magnetic field measurement system 140 and stimulus device 182, when separate, can be in communication with each other. The communication can be direct communication or can be indirect communication through a network 180 (for example, a local area network, a wide area network, the Internet, or any combination thereof). Methods of direct or indirect communication can include wired or wireless (e.g., RF, optical, Wi-Fi, Bluetooth™, or infrared or the like) communications methods or any combination thereof. The analysis or application device 184, when present, may be in direct or indirect communication with the magnetic field measurements system 140 and stimulus device 182.

FIG. 2 shows the magnetic spectrum from 1 fT to 100 μT in magnetic field strength on a logarithmic scale. The magnitude of magnetic fields generated by the human brain are indicated by range 201 and the magnitude of the background ambient magnetic field, including the Earth's magnetic field, by range 202. The strength of the Earth's magnetic field covers a range as it depends on the position on the Earth as well as the materials of the surrounding environment where the magnetic field is measured. Range 210 indicates the approximate measurement range of a magnetometer (e.g., an OPM) operating in the SERF mode (e.g., a SERF magnetometer) and range 211 indicates the approximate measurement range of a magnetometer operating in a scalar mode (e.g., a scalar magnetometer.) Typically, a SERF magnetometer is more sensitive than a scalar magnetometer but many conventional SERF magnetometers typically only operate up to about 0 to 200 nT while the scalar magnetometer starts in the 10 to 100 fT range but extends above 10 to 100 μT.

Electroencephalography (EEG) instruments have been used to obtain neural signals from the brain. In many instances, EEG instruments are hampered by poor spatial resolution of the EEG signal. The EEG signal is also susceptible to low signal-to-noise ratio (SNR) and artifacts detected from the skull and other brain tissue (e.g., if the user wiggles his or her eyebrows, blinks, or performs any number of other head movements).

Functional magnetic resonance imaging (fMRI) instruments have also been used to obtain haemodynamic signals from the brain that are related to neural signals. However, these instruments require large magnets enclosed within a tunnel tube-type enclosure that patients lie within which are known to cause claustrophobia, and thus, cannot be scaled to wearable or portable form factors.

Superconducting quantum interference device (SQUID)-based magnetoencephalography (MEG) instruments have also been used to obtain neural signals from the brain. However, SQUID-based MEG systems suffer from lack of mobility, particularly in portable head-wearable devices, since these systems require cryogenic cooling, including a lot of maintenance making the system prohibitively costly for a user.

In contrast, an optically pumped magnetometer (OPM)-based MEG system allows for a large range of motion, portability, and head movement. Examples of such systems are described in U.S. Patent Application Publications Nos. 2020/0072916; 2020/0056263; 2020/0025844; 2020/0057116; 2019/0391213; 2020/0088811; 2020/005711; 2020/0109481; and 2020/0123416; U.S. patent application Ser. Nos. 16/573,394; 16/573,524; 16/679,048; 16/741,593; 16/752,393; 16/850,380 and 16/850,444, and U.S. Provisional Patent Application Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; and 62/983,406, all of which are incorporated herein by reference in their entireties.

Further details discussing different form factors in small, portable, wearable devices and applications thereof are set forth in U.S. patent application Ser. Nos. 16/523,861; 16/457,655; and Ser. No. 16/364,338, and U.S. Provisional Patent Application Ser. Nos. 62/752,067; 62/829,124; 62/839,405; 62/894,578; 62/859,880; and 62/891,128, all of which are incorporated herein by reference in their entireties.

Methods and systems are described herein to differentiate different types of sensory input using signal envelope modulation which, at least in some embodiments, is imperceptible or near imperceptible. The envelope modulation may be a single-frequency or broadband (for example, noise) modulation of any suitable width and shape.

In at least some embodiments, the methods and systems utilize the response of early sensory cortices to stimulation in the visual, auditory, or somatosensory domain. In at least some embodiments, that response may be linear. Stimulus tags, such as frequency tags or broadband (for example, noise) tags can be placed over portions of stimulus to produce tagging. For example, the stimulus tag can be placed over portions of a fixed or moving image to provide visual tagging, over portions of a sound to provide auditory tagging, or over an area of the body to provide haptic or tactile tagging. The image may be, for example, a picture, movie, television show, advertisement, video clip, or a video game. The sound may be, for example, a song, podcast, advertisement, or auditory signal from a movie, television show, video clip, or video game. The tactile stimulus over an area of the body can be provided by a haptic arrangement or moving device (such as a video game controller or mobile device).

Any suitable stimulus tag that can be determined from the received magnetic field signals can be used. A frequency tag can be periodic stimulus or variation at a specific frequency (for example, 6 Hz or 15 Hz or the like). A broadband or noise tag can be semi-random or fully random, wide-band stimulus or variation. In at least some embodiments, for a broadband tag short and long pulses can be mixed to generate a broadband signal ranging from, for example, 0 to 150 Hz. In at least some embodiments, the broadband tag can be manipulated to remove the lower end of the range to have a tag ranging from, for example, 40 to 100 Hz.

FIGS. 3-5 illustrate one example of one embodiment of a frequency-tagging technique in the visual domain. FIG. 3 is a graph of the power spectral density (PSD) of spatially-filtered MEG data obtained in response to visual stimulation modulated by a frequency tag at three different frequencies. Curve 102 corresponds to visual stimulation modulated by a frequency tag at 6 Hz, curve 104 corresponds to visual stimulation modulated by a frequency tag at 10 Hz, and curve 106 corresponds to visual stimulation modulated by a frequency tag at 15 Hz. In these embodiments, the visual stimulus was a radial grating 190 (see insert in FIG. 3) presented at the center of the screen for 10 s per trial. The PSD was computed separately for each trial (10s), and each curve 102, 104, 106 represents the median across trials for each condition (about 20 trials per condition.) It can be seen for example that 6 Hz visual stimulation leads to large peaks in curve 102 at 6 Hz (main frequency) and its first and second harmonics (12 Hz, 18 Hz). This PSD is for a weighted combination of actual MEG channels, which was trained on independent data using Canonical Correlation Analysis (CCA). In FIG. 3, only frequencies up to 100 Hz are shown, but, in at least some embodiments, the MEG system can reliably record brain signals at higher frequencies, up to, for example, approximately 200 Hz.

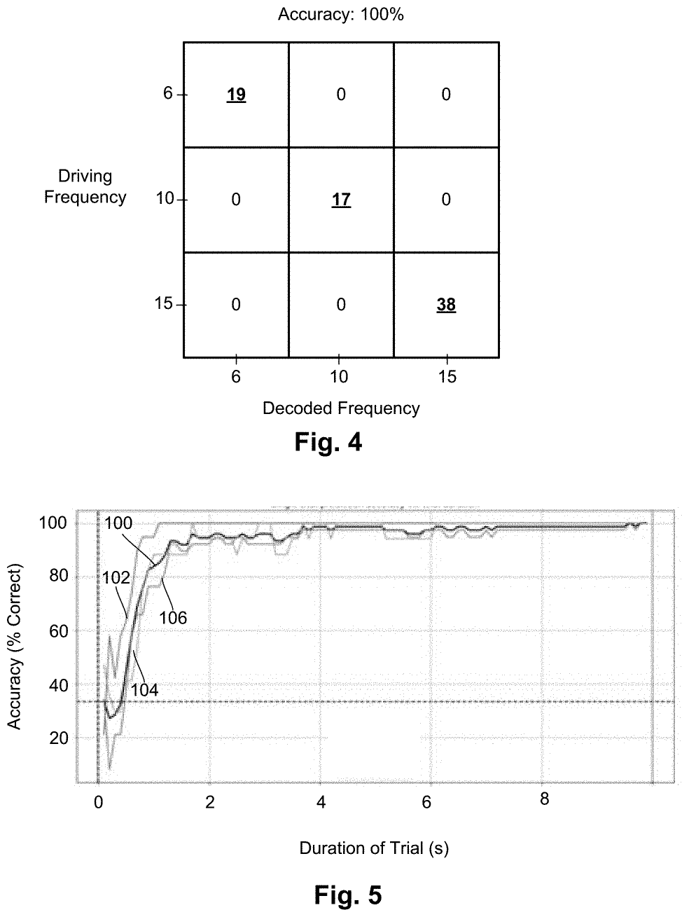

FIGS. 4 and 5 illustrate decoding accuracy for the same frequency tagging dataset used for FIG. 3. As in FIG. 3, curve 102 corresponds to visual stimulation modulated by a frequency tag at 6 Hz, curve 104 corresponds to visual stimulation modulated by a frequency tag at 10 Hz, and curve 106 corresponds to visual stimulation modulated by a frequency tag at 15 Hz. Curve 100 corresponds to the accuracy for all conditions.

Using a spatial filter trained with CCA, and correlating the combined brain signals with the candidate frequencies, the dataset used for FIGS. 3-5 provides for prediction of which of three frequencies was presented with 100% accuracy for a 10-second-long trial, as illustrated in FIG. 4. Performance of the classifier is high for much shorter trials, asymptoting as early as approximately 1.5 s in this dataset, as illustrated in FIG. 5.

To obtain the dataset used to prepare FIGS. 3-5, a subject was equipped with an array of eight OPM sensors on the back of the head, and watched the flickering radial stimulus 190 (FIG. 3) for 10 seconds per trial, with a total of 80 trials. The flicker was periodic, at 6, 10, or 15 Hz for any given trial. The power spectral density averaged per condition shows a very strong response to the driving frequency and its harmonics. A time-domain decoder, trained on a few trials, can identify the driving frequency for any given 10 second trial (as illustrated in FIG. 4), and this performance can be achieved with trials as short as 2 seconds for this subject and dataset.

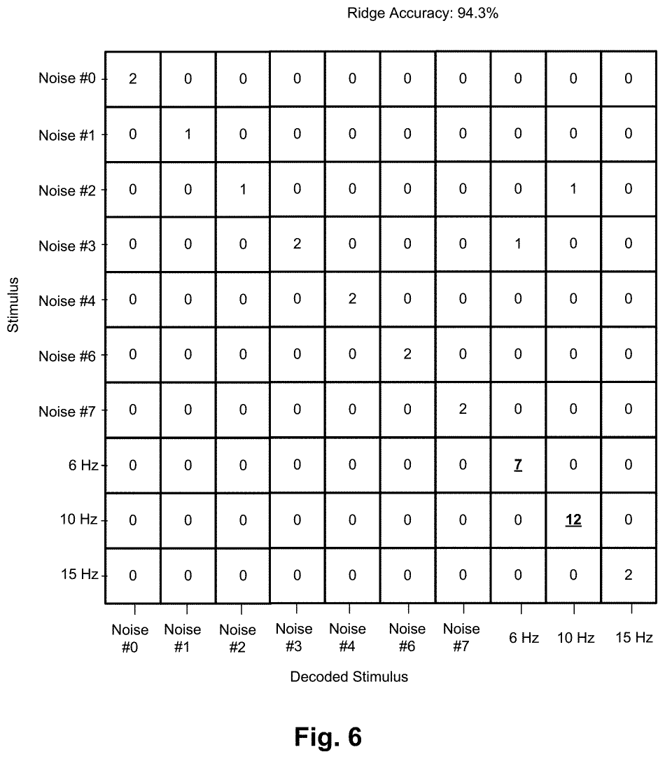

FIG. 6 illustrates classification accuracy for another example of an embodiment in which the same subject was shown, during different trials, not only the three frequency tags, but also several semi-random sequences (noise tag modulation) of flashes of the radial stimulus. FIG. 6 is a confusion matrix for the single-trial decoding of short, 2 second visual tags (both frequency tags and noise tags). A deconvolution algorithm, based on a ridge regression classifier, can predict which of 10 codes was shown to the subject with high accuracy. A classifier trained to learn a deconvolution filter (based on the first 10 trials of the experiment) can decode the visual pattern shown to the subject with great accuracy. Hence frequency tagging and noise tagging give rise to strong signals and accurate decoding.

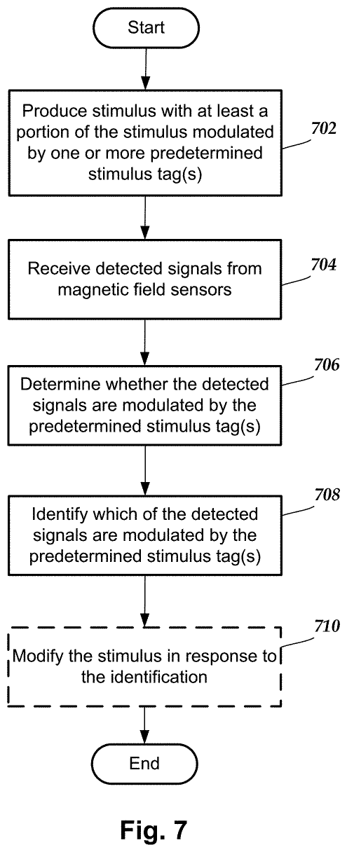

FIG. 7 is a flowchart of one embodiment of a method or system for identifying biological signals that are modulated by one or more predetermined stimulus tags. In step 702, a stimulus is produced or provided to a user. At least a portion of the stimulus is modulated by one or more predetermined stimulus tags. For example, the stimulus can be a visual stimulus (e.g., a picture, movie, television show, advertisement, video clip, or a video game), an auditory stimulus (e.g., a song, podcast, or auditory signal from a movie, television show, advertisement, video clip, or video game), or a tactile stimulus (for example, from a haptic arrangement associated with a video game, movie, television show, or the like) or any combination thereof. The stimulus may be produced or provided by a magnetic field measurement system (or biological signal detection system) or may be provided by another system, as illustrated in FIGS. 1A and 1C. The entire stimulus can be modulated or only a portion of the stimulus can be modulated such as, for example, a region on the displayed picture, movie, television show, or video game or a particular track or voice of an auditory stimulus. Each stimulus tag can be, for example, a frequency tag, a broadband tag, or a noise tag.

In at least some embodiments, when multiple stimulus tags are used, the portions of the stimulus that are modulated by different stimulus tags may be spatially or temporally separated. For example, different portions of a visual stimulus may be modulated with different stimulus tags. In at least some embodiments, single frequency, broadband, or noise modulation may be applied to a portion or different portions of the stimulus, for example, an image or moving image (video or movie). As examples, different modulation sequences may spatially encode different regions of interest, characters, or features of the fixed or moving image. Similarly, different modulation sequences can be overlaid into different segments of an auditory signal. In at least some embodiments, the modulation for an auditory stimulus could be a broadband auditory signal, like a chirp, that is introduced into fast sequences of heard sounds to, for example, decode how the brain handles contrast in changes of heard sound. The stimulus tag could also be an overall modulation of the auditory stream or of competing auditory streams with different frequencies. In at least some embodiments, auditory tags can be natural, binaural stimuli that the subject does not perceive or can be intentional insertions that strategically manipulate magnitude and phase of the auditory stream.

In step 704, biological signals arising from magnetic field sources (for example, neural sources in the brain of a user or other biological sources) are detected by one or more magnetic field sensors and these detected signals are received by a processor of the magnetic field measurement system or other analysis or application device (see, for example, FIGS. 1A and 1C). The magnetic field sensors can be OPMs of a magnetic field measurement system (for example, a biological signal detection system) such as those illustrated in FIGS. 1A and 1B or any other suitable magnetic field sensors.

In step 706, the processor determines whether the detected signals are modulated by the predetermined stimulus tag(s). For example, the detected signals can be correlated to the predetermined stimulus tag(s) (under the simplifying assumption that the brain operates as a Linear Time Invariant system at this scale of investigation), and high correlation values can be indicative of the modulation. Low correlation values can be indicative of the lack of modulation. In at least some embodiments, the detected signals from multiple sensors can be combined optimally to enhance the correlation between the predetermined stimulus tags and the detected signals (for example, Canonical Correlation Analysis may be used for such purposes). In at least some embodiments, a power spectrum can be generated, as illustrated in FIG. 3. Time-domain analysis of the signals can also be used. In at least some embodiments, determining whether the detected signals are modulated by the predetermined stimulus tag comprises generating a correlation between the detected signals and the predetermined stimulus tag and determining whether the detected signals are modulated by the predetermined stimulus tag. In at least some embodiments, determining whether the detected signals are modulated by the predetermined stimulus tag includes time series correlation of the detected signal to the determined stimulus tag.

As an example, detected signals from a single OPM sensor (or a subset of OPM sensors or even a combination of all of the OPM sensors) can be combined and used to generate correlation values with the predetermined stimulus tag(s) corresponding to that OPM sensor (or subset of OPM sensors or all OPM sensors). When only using a single OPM sensor or subset of OPM sensors the resulting correlation values can identify spatial variations in the modulation. When generating correlation values using detected signals from different time periods, temporal variations in the modulation can be determined. Such temporal variations may be indicative of a change in focus of the user on different portions of the stimulus or changes in the stimulus or modulation itself.

In step 708, those detected signals that are modulated by the stimulus tag(s) are identified for further analysis or for any other suitable application or use.

In step 710, the identification of the modulation by the stimulus tag(s) in the detected signals can be optionally used to modify the stimulus or provide a new stimulus in response. Examples of applications of modification of stimuli are presented below. The modification may be produced by a magnetic field measurement system (or biological signal detection system) or may be produced by a stimulus system or device, as illustrated in FIGS. 1A and 1C.

In at least some embodiments, multiple stimulus tags can be used to determine or monitor whether the user's attention is on multiple stimuli. In at least some embodiments, the method or system can be used to provide a closed-loop MEG-brain machine interface in which the stimulus influences the brain signals which are detected using stimulus tag(s), as described above, and the system or method can then modify the stimulus or provide a new stimulus in response. In at least some embodiments, steps 702-710, shown in FIG. 7, can be repeated one or more times (for example, periodically or continuously) to detect signals from magnetic field sensors and to continue modifying the stimulus, or providing a new stimulus, in response to the identification of the stimulus tag(s) using the closed-loop MEG-brain machine interface.

There are a variety of possible applications using stimulus tags and the determination and identification of the stimulus tags in detected biological signals. As an example, MEG measurements of a subject watching fixed or moving images, or listening to an auditory signal, or otherwise engaged in a task involving early sensory cortices, may be employed to determine localization of a subject's attention, overtly or covertly, by tagging one or more portions of the stimulus.

One example of an area for application is in video games. For example, one or more portions of a displayed video game can be modulated using a visual or auditory tag. The biological signal detection system can be used to determine whether signals generated by magnetic field sources in the brain of the player are modulated by the visual or auditory tag. This can be used to monitor or modify the video game. For example, by measuring a player's spatial attention, new story lines or tactical situations may be created in portions of the scenery a player is not paying attention to, making the game more unpredictable, challenging, and interesting.

Another area for application is neuromarketing. By stimulus tagging different portions, features, or characters of a pictorial or video commercial, marketing elements may be designed to determine whether a subject's attention is focused on the product.

A further area of application is education. Stimulus tagging may be used to determine whether a student's attention is focused on educational content.

Yet another area of application is ophthalmology and optometry. Stimulus tagging may be used to objectively determine blind spots in the visual field and the span of peripheral vision. These tagging techniques may be combined with eye tracking.

Another application is biometric screening. Stimulus tagging combined with source localization may be used to map the primary visual cortex. Individuals may have unique visual cortex characteristics.

Further applications include detection of drug or alcohol impairment; detection or determination of ADHD (attention deficient hyperactivity disorder—for example, determine how long someone can stay focused on something); and autism spectrum disorder screening (for example, detection of repetitive focus on certain objects).

A further application is decoding feedback. The brain's inclusion of the stimulus tag can be decoded by a MEG signal to predict attention, memory, or other cognitive functions. These predictions can be utilized to adjust visual or auditory signal to better suit the user. For example, when listening to multiple streams of audio that are tagged, identifying a stimulus tag in the generated neural magnetic field signals can predict which of the audio streams the subject is paying more attention to and increase the salience of that audio stream. A similar embodiment can be applied to visual stimulus.

In at least some embodiments, portions of an image may be modulated with stimulus tags to enable determination of the spatial location of a subject's attention using MEG signals. In at least some embodiments, feedback algorithms can decode the brain's encoding of the stimulus tags and adjust the stimulus in the subject's environment to better suit their attention, memory, or overall cognition.

In at least some embodiments, advantages of the use of stimulus tags, as described herein, over EEG monitoring can include spatial resolution and higher fidelity processes to acquire neural signals from a user. For example, in at least some embodiments, the MEG signal can be confined to smaller segments of the brain, allowing for the use of tagging with more of the user's environment/stimuli and interpretation of a larger range of stimulus tags. In addition, the MEG signal may include more content and higher dimensional information in less signal.

In at least some embodiments, advantages over SQUID MEG systems can include permitting head and body movement; customized magnetic field sensor placement on the user's head; and the positioning of magnetic field sensors closer to the user's brain.

The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification provides a description of the invention and its manufacture and use. Since many embodiments of the invention can be made without departing from the spirit and scope of the invention, the invention also resides in the claims hereinafter appended.

What is claimed as new and desired to be protected by Letters Patent of the United States is:

1. A biological signal detection system, comprising:
one or more magnetic field sensors configured and arranged for placement on a user and for detecting signals generated by biological magnetic field sources of the user;
at least one memory;
at least one processor coupled to the at least one memory and the one or more magnetic field sensors and configured to receive the detected signals of the one or more magnetic field sensors, wherein the at least one processor is configured to perform actions comprising:
receiving the detected signals from the magnetic field sensors;
determining whether the detected signals are modulated by a predetermined stimulus tag; and
in response to the determination, identifying which of the detected signals are modulated by the predetermined stimulus tag.

2. The biological signal detection system of claim 1, wherein the actions further comprise
producing a stimulus for the user; and
modulating a portion of the stimulus using the predetermined stimulus tag.

3. The biological signal detection system of claim 2, wherein the actions further comprise
altering the stimulus in response to identifying which of the detected signals are modulated by the predetermined stimulus tag.

4. The biological signal detection system of claim 3, wherein altering the stimulus comprises altering a visual content of the stimulus in response to the determination.

5. The biological signal detection system of claim 2, wherein the stimulus is a visual stimulus.

6. The biological signal detection system of claim 2, wherein the stimulus is an audible stimulus.

7. The biological signal detection system of claim 2, wherein the stimulus is a tactile stimulus.

8. The biological signal detection system of claim 1, wherein the stimulus tag is a frequency tag at a predetermined frequency.

9. The biological signal detection system of claim 1, wherein the stimulus tag is a broadband tag over a predetermined range of frequencies.

10. The biological signal detection system of claim 1, wherein the stimulus tag is a noise tag.

11. The biological signal detection system of claim 1, wherein determining whether the detected signals are modulated by the predetermined stimulus tag comprises generating a correlation between the detected signals and the predetermined stimulus tag and determining whether the detected signals are modulated by the predetermined stimulus tag.

12. The biological signal detection system of claim 1, wherein determining whether the detected signals are modulated by the predetermined stimulus tag comprises determining whether the detected signals are modulated by a predetermined first stimulus tag;
wherein identifying which of the detected signals are modulated by the predetermined stimulus tag comprises identifying which of the detected signals are modulated by the predetermined first stimulus tag;
wherein the actions further comprise
determining whether the detected signals are modulated by a predetermined second stimulus tag, wherein the second stimulus tag is different from the first stimulus tag; and
in response to the determination, identifying which of the detected signals are modulated by the predetermined second stimulus tag.

13. The biological signal detection system of claim 1, wherein the actions further comprise communicating with a stimulus generator indicating which of the detected signals are modulated by the predetermined stimulus tag.

14. The biological signal detection system of claim 1, wherein the detected signals originate in a brain of a user.

15. The biological signal detection system of claim 2, wherein the biological signal detection system is a closed-loop MEG-brain machine interface.

16. The biological signal detection system of claim 12, wherein the biological signal detection system is a closed-loop MEG-brain machine interface.

17. A non-transitory computer-readable medium having stored thereon instructions for execution by a processor to perform actions including:
receiving detected signals from one or more magnetic field sensors;
determining whether the detected signals are modulated by a predetermined stimulus tag; and
in response to the determination, identifying which of the detected signals are modulated by the predetermined stimulus tag.

18. The non-transitory computer-readable medium of claim 17, wherein the actions further include
producing a stimulus for a user; and
modulating a portion of the stimulus using the predetermined stimulus tag.

19. The non-transitory computer-readable medium of claim 18, wherein the actions further include altering the stimulus in response to identifying which of the detected signals are modulated by the predetermined stimulus tag.

20. The non-transitory computer-readable medium of claim 17, wherein the stimulus tag is a frequency tag at a predetermined frequency.

21. The non-transitory computer-readable medium of claim 17, wherein the stimulus tag is a broadband tag over a predetermined range of frequencies.

22. The non-transitory computer-readable medium of claim 17, wherein the stimulus tag is a noise tag.
